(19) United States
(12) Patent Application Publication
(10) Pub. No.: US 2024/0273351 A1
(43) Pub. Date: Aug. 15, 2024

(54) METHOD OF GENERATING NEGATIVE SAMPLE SET FOR PREDICTING MACROMOLECULE-MACROMOLECULE INTERACTION, METHOD OF PREDICTING MACROMOLECULE-MACROMOLECULE INTERACTION, METHOD OF TRAINING MODEL, AND NEURAL NETWORK MODEL FOR PREDICTING MACROMOLECULE-MACROMOLECULE INTERACTION

(71) Applicant: BOE Technology Group Co., Ltd., Beijing (CN)

(72) Inventor: Zhenzhong Zhang, Beijing (CN)

(73) Assignee: BOE Technology Group Co., Ltd., Beijing (CN)

(21) Appl. No.: 17/907,503

(22) PCT Filed: Dec. 30, 2021
(86) PCT No.: PCT/CN2021/142904

§ 371 Date: Jan. 31, 2023

(51) Int. Cl. G06N 3/08; G06N 5/022; G16B 5/00; G16B 40/20

(52) U.S. Cl. CPC G06N 3/08; G06N 5/022; G16B 5/00; G16B 40/20

(57) ABSTRACT

A method of generating a negative sample set for predicting macromolecule-macromolecule interaction is provided. The method includes receiving a positive sample set including pairs of macromolecules of a first type and macromolecules of a second type having macromolecule-macromolecule interaction; generating a first similarity map of the macromolecules of the first type; generating a second similarity map of the macromolecules of the second type; generating vectorized representations of nodes in the first similarity map and vectorized representations of nodes in the second similarity map; and generating the negative sample set using the vectorized representations of nodes in the first similarity map and the vectorized representations of nodes in the second similarity map.

### TECHNICAL FIELD

The present invention relates to machine learning technology, more particularly, to a method of generating a negative sample set for predicting macromolecule-macromolecule interaction, a method of predicting macromolecule-macromolecule interaction, a method of training a model for generating a negative sample set for predicting macromolecule-macromolecule interaction, and a neural network model for predicting macromolecule-macromolecule interaction.

### BACKGROUND

Protein-RNA interaction plays important roles in various processes in a cell, including posttranscriptional regulation of gene expression, protein translation, RNA post-transcriptional modification, and cellular regulation. In recent years, a lot of efforts have been placed on prediction of protein-RNA interaction. Typically, the prediction methods are based on structures, chemical properties, and biological functions of RNA molecules and protein molecules.

### SUMMARY

In one aspect, the present disclosure provides a method of generating a negative sample set for predicting macromolecule-macromolecule interaction, comprising receiving a positive sample set comprising pairs of macromolecules of a first type and macromolecules of a second type having macromolecule-macromolecule interaction; generating a first similarity map of the macromolecules of the first type; generating a second similarity map of the macromolecules of the second type; generating vectorized representations of nodes in the first similarity map and vectorized representations of nodes in the second similarity map; and generating the negative sample set using the vectorized representations of nodes in the first similarity map and the vectorized representations of nodes in the second similarity map.

Optionally, the first similarity map or the second similarity map comprises nodes and edges connecting adjacent nodes, wherein a respective node represents a respective macromolecule, a respective edge represents a respective distance between a respective pair of the macromolecules, and a respective weight of the respective edge represents a respective similarity between the respective pair of the macromolecules.

Optionally, the method further comprises generating a plurality of intermediate sets; wherein the positive sample set is represented by {(m1ᵢ, m2ᵢ), i=1, . . . , K}, wherein m1ᵢ stands for an i-th macromolecule of the first type and m2ᵢ stands for an i-th macromolecule of the second type; wherein generating the respective intermediate set of the plurality of intermediate sets comprises sorting m1ⱼ (j=1, . . . , K, and j≠i) based on similarities between m1ᵢ and m1ⱼ to obtain a subset of m1ⱼ (j=1, . . . , K, and j≠i); determining a probability of interaction between m2ᵢ and each sample in the subset; and generating the respective intermediate set based on the probability of interaction.

Optionally, the method further comprises calculating similarities between m1ᵢ and m1ⱼ (j=1, . . . , K, and j≠i) by:

$$S ( {dr}_{j} , {dr}_{i} ) = \frac{〈 {dr}_{j} , {dr}_{i} 〉}{\sqrt{〈 {dr}_{j} , {dr}_{j} 〉} \times \sqrt{〈 {dr}_{i} , {dr}_{i} 〉}} ;$$

wherein drⱼ stands for vectorized representations of nodes in the subset; and drᵢ stands for a vectorized representation of m1ᵢ.

Optionally, the probability of interaction between m2ᵢ and each sample in the subset is determined by:

$$P ( 1 \vert {dr}_{j} , {dp}_{i} ) = \frac{1}{1 + exp ( - θ \cdot [ {dr}_{j} , {dp}_{i} , {dr}_{j} - {dp}_{i} , {dr}_{j} ⊗ {dp}_{i} ] )} ;$$

wherein drⱼ stands for vectorized representations of nodes in the subset; dpᵢ stands for vectorized representations of m2ᵢ; P(1|drⱼ, dpᵢ) stands for a probability of interaction between m2ᵢ and each sample in the subset; [,] stands for stitching between elements; ⊗ stands for product of two vectors; and θ stands for a parameter that is tunable.

Optionally, the method further comprises placing (m1ⱼ, 1−P(1|drⱼ, dpᵢ)) into the respective intermediate set when P(1|drⱼ, dpᵢ) is less than a threshold value.

Optionally, sampling L number of negative samples from the respective intermediate set is performed based on probability {pₖ, k=1, . . . , |T|};
wherein

$${p}_{k} = \frac{1 - P ( 1 \vert {dr}_{j} , {dp}_{i} )}{{\sum }_{l = 1}^{\vert T \vert} ( 1 - P ( 1 \vert {dr}_{j} , {dp}_{i} ) )} ;$$

   |T| stands for a number of elements in the respective intermediate set; and (m1ⱼ, 1−P(1|drⱼ, dpᵢ)) stands for a k-th element in the intermediate set.

Optionally, generating the negative sample set comprises sampling L number of negative samples from a respective intermediate set of the plurality of intermediate sets; wherein the negative sample set comprises negative samples sampled from the plurality of intermediate sets; and L is an integer equal to or greater than 1.

Optionally, the macromolecules of the first type comprise RNA molecules and macromolecules of the second type comprise protein molecules.

In another aspect, the present disclosure provides a method of predicting macromolecule-macromolecule interaction using the positive sample set and the negative sample set generated by the method of generating a negative sample set described herein.

In another aspect, the present disclosure provides a method of training a model for generating a negative sample set for predicting macromolecule-macromolecule interaction, comprising receiving a positive sample set comprising pairs of macromolecules of a first type and macromolecules of a second type having macromolecule-macromolecule interaction; generating a first similarity map of macromolecules of a first type; generating a second similarity map of macromolecules of a second type; generating vectorized representations of nodes in the first similarity map and vectorized representations of nodes in the second similarity map; determining a probability of interaction between a first respective vectorized representation of a node in the first similarity map and a second respective vectorized representation of a node in the second similarity map; and training the model at least partially based on the probability of interaction.

Optionally, the probability of interaction is determined by:

$$P ( 1 \vert dm {1}_{i} , dm {2}_{j} ) = \frac{1}{1 + exp ( - θ \cdot [ dm {1}_{i} , dm {2}_{j} , dm {1}_{i} - dm {2}_{j} , dm {1}_{i} ⊗ dm {2}_{j} ] )} ;$$

wherein dm1ᵢ stands for vectorized representations of nodes in the first similarity map; dm2ⱼ stands for vectorized representations of nodes in the second similarity map; p(1|dm1ᵢ, dm2ᵢ) stands for a probability of interaction between a first respective vectorized representation of a node in the first similarity map and a second respective vectorized representation of a node in the second similarity map; [,] stands for stitching between elements; and ⊗ stands for product of two vectors; and θ stands for a parameter that is tunable.

Optionally, the first similarity map or the second similarity map comprises nodes and edges connecting adjacent nodes, wherein a respective node represents a respective macromolecule, a respective edge represents a respective distance between a respective pair of the macromolecules, and a respective weight of the respective edge represents a respective similarity between the respective pair of the macromolecules.

Optionally, a respective similarity between a respective pair of the macromolecules of the first type is expressed as:

$$sim 1 ( {m}_{1 - 1} , {m}_{1 - 2} ) = 1 - d 1 ( {m}_{1 - 1} , {m}_{1 - 2} ) ;$$

wherein (m₁₋₁, m₁₋₂) stands for the respective pair of the macromolecules of the first type, sim1 stands for the respective similarity between the respective pair of the macromolecules of the first type, and d1 stands for a distance between the respective pair of the macromolecules of the first type.

Optionally, d1 is expressed as:

$$d 1 ( {m}_{1 - 1} , {m}_{1 - 2} ) = \frac{lev ( {m}_{1 - 1} , {m}_{1 - 2} )}{max ( len ( {m}_{1 - 1} ) , len ( {m}_{1 - 2} ) )} ;$$

wherein lev(m₁₋₁, m₁₋₂) stands for an edit distance between the respective pair of the macromolecules of the first type, len(m₁₋₁) stands for a length of a first macromolecule of the first type in the respective pair, and len(m₁₋₂) stands for a length of a second macromolecule of the first type in the respective pair.

Optionally, a respective similarity between a respective pair of the macromolecules of the second type is expressed as:

$$sim 2 ( {m}_{2 - 1} , {m}_{2 - 2} ) = 1 - d 2 ( {m}_{2 - 1} , {m}_{2 - 2} ) ;$$

wherein (m₂₋₁, m₂₋₂) stands for the respective pair of the macromolecules of the second type, sim2 stands for the respective similarity between the respective pair of the macromolecules of the second type, and d2 stands for a distance between the respective pair of the macromolecules of the second type.

Optionally, d2 is expressed as:

$$d 2 ( {m}_{2 - 1} , {m}_{2 - 2} ) = \frac{lev ( {m}_{2 - 1} , {m}_{2 - 2} )}{max ( len ( {m}_{2 - 1} ) , len ( {m}_{2 - 2} ) )} ;$$

wherein lev(m₂₋₁, m₂₋₂) stands for an edit distance between the respective pair of the macromolecules of the second type, len(m₂₋₁) stands for a length of a first macromolecule of the second type in the respective pair, and len(m₂₋₂) stands for a length of a second macromolecule of the second type in the respective pair.

Optionally, the first similarity map includes N1 number of nodes, {eᵢ, i=1, . . . , N1}; and M1 number of edges, {rⱼ, j=1, . . . , M}; a respective vectorized representation of a respective node in the first similarity map is expressed as:

$${h}^{t 1 + 1} ( {e}_{i} ) = σ ( {W}_{p} \times {h}^{t 1} ( {e}_{i} ) + {\sum }_{{e}_{k} ∈ N ( {e}_{i} )} {W}_{ph} \times {h}^{t 1} ( {e}_{k} ) ) ;$$

wherein eᵢ stands for a respective node in the first similarity map; h^{t1}(eᵢ) stands for a respective vectorized representation of the respective node eᵢ prior to a t1-th step reiteration; h^{t1+1}(eᵢ) stands for an updated respective vectorized representation of the respective node eᵢ subsequent to the t1-th step reiteration; σ stands for a leaky relu activation function; N(eᵢ) stands for a set of nodes neighboring the respective node eᵢ; and Wₚ, Wₚₕ stand for parameters of a graph neural network for generating the vectorized representation.

Optionally, the second similarity map includes N2 number of nodes, {e′i, i=1, . . . , N2}; and M2 number of edges, {r′ⱼ, j=1, . . . , M2}; a respective vectorized representation of a respective node in the second similarity map is expressed as:

$${h}^{t 2 + 1} ( {e}_{i}^{′} ) = σ ( {\sum }_{{e}_{k}^{′} ∈ N ( {e}_{i}^{′} ) ⋃ {e}_{i}^{′}} {α}_{i , k}^{t 2 + 1} {W}_{p}^{t + 1} \times {h}^{t 2} ( {e}_{k}^{′} ) ) ; \\ {α}_{i , k}^{t 2 + 1} = softmax ( 〈 {h}^{t 2} ( {e}_{i}^{′} ) , {h}^{t 2} ( {e}_{k}^{′} ) 〉 ) ;$$

wherein e′ᵢ stands for a respective node in the second similarity map; h^{t2}(e′ᵢ) stands for a respective vectorized representation of the respective node e′ᵢ prior to a t2-th step reiteration; h^{t2+1}(e′ᵢ) stands for an updated respective vectorized representation of the respective node e′ᵢ subsequent to the t2-th step reiteration; σ stands for a leaky relu activation function; N(e′ᵢ) stands for a set of nodes neighboring the respective node e′ᵢ; <h^{t2}(eᵢ′), h^{t2}(eₖ′)> stands for an inner product of h^{t2}(eᵢ′) and h^{t2}(eₖ′); Wₚ^{t2+1} stands for a parameter of a graph neural network for generating the vectorized representation; and α_{i,k}^{t2+1} stands for attention weights representing a link strength between node eᵢ′ and node eₖ′.

Optionally, the positive sample set is represented by {(m1ᵢ, m2ᵢ), i=1, . . . , K}, wherein m1ᵢ stands for an i-th macromolecule of the first type and m2ᵢ stands for an i-th macromolecule of the second type; wherein training the model comprises minimizing a loss function:

$$L = - {\sum }_{i = 1}^{K} log p ( 1 \vert dm {1}_{i} , dm {2}_{i} ) ;$$

wherein dm1ᵢ stands for vectorized representations of nodes in the first similarity map; dm2ⱼ stands for vectorized representations of nodes in the second similarity map; p(1|dm1ᵢ, dm2ᵢ) stands for a probability of interaction between a first respective vectorized representation of a node in the first similarity map and a second respective vectorized representation of a node in the second similarity map.

Optionally, the macromolecules of the first type comprise RNA molecules and macromolecules of the second type comprise protein molecules.

In another aspect, the present disclosure provides a neural network model for predicting macromolecule-macromolecule interaction, trained by the method of training a model described herein.

### BRIEF DESCRIPTION OF THE FIGURES

The following drawings are merely examples for illustrative purposes according to various disclosed embodiments and are not intended to limit the scope of the present invention.

FIG. 1 illustrates a process of generating negative samples for predicting macromolecule-macromolecule interaction.

FIG. 2 illustrates a process of generating negative samples for predicting macromolecule-macromolecule interaction.

FIG. 3 illustrates a process of generating negative samples for predicting macromolecule-macromolecule interaction.

FIG. 4 illustrates a process of training a model for generating a negative sample set for predicting macromolecule-macromolecule interaction in some embodiments according to the present disclosure.

FIG. 5 illustrates a process of generating a negative sample set for predicting macromolecule-macromolecule interaction in some embodiments according to the present disclosure.

FIG. 6 illustrates a specific example of generating a negative sample set for predicting macromolecule-macromolecule interaction in some embodiments according to the present disclosure.

FIG. 7 is a schematic diagram of a structure of an apparatus in some embodiments according to the present disclosure.

### DETAILED DESCRIPTION

The disclosure will now be described more specifically with reference to the following embodiments. It is to be noted that the following descriptions of some embodiments are presented herein for purpose of illustration and description only. It is not intended to be exhaustive or to be limited to the precise form disclosed.

The present disclosure provides, inter alia, a method of generating a negative sample set for predicting macromolecule-macromolecule interaction, a method of predicting macromolecule-macromolecule interaction, a method of training a model for generating a negative sample set for predicting macromolecule-macromolecule interaction, and a neural network model for predicting macromolecule-macromolecule interaction that substantially obviate one or more of the problems due to limitations and disadvantages of the related art. In one aspect, the present disclosure provides a method of generating a negative sample set for predicting macromolecule-macromolecule interaction. In some embodiments, the method includes receiving a positive sample set comprising pairs of macromolecules of a first type and macromolecules of a second type having macromolecule-macromolecule interaction; generating a first similarity map of the macromolecules of the first type; generating a second similarity map of the macromolecules of the second type; generating vectorized representations of nodes in the first similarity map and vectorized representations of nodes in the second similarity map; and generating the negative sample set using the vectorized representations of nodes in the first similarity map and the vectorized representations of nodes in the second similarity map. As used herein, the term “sample set” may include one or more samples. In one example, the sample set (e.g., the negative sample set or the positive sample set) includes a single sample. In another example, the sample set (e.g., the negative sample set or the positive sample set) includes multiple samples.

As used herein, the term “macromolecule” refers to any protein, ribonucleic acid (RNA), deoxyribonucleic acid (DNA), carbohydrate, polypeptide, polynucleotide, and other large biomolecules.

Using computational models for predicting macromolecule-macromolecule interaction (e.g., protein-RNA interaction) typically requires positive samples and negative samples. Positive samples include known macromolecule pairs (e.g., protein-RNA pairs) based on experiments. However, experimentally validated negative samples are difficult to find. Although negative samples may be randomly generated, the quality of these negative samples cannot be guaranteed as they unavoidably include false negative sample. Because the quality of negative samples is unsatisfactory, prediction performance of computational models using these randomly generated negative samples is at best unreliable.

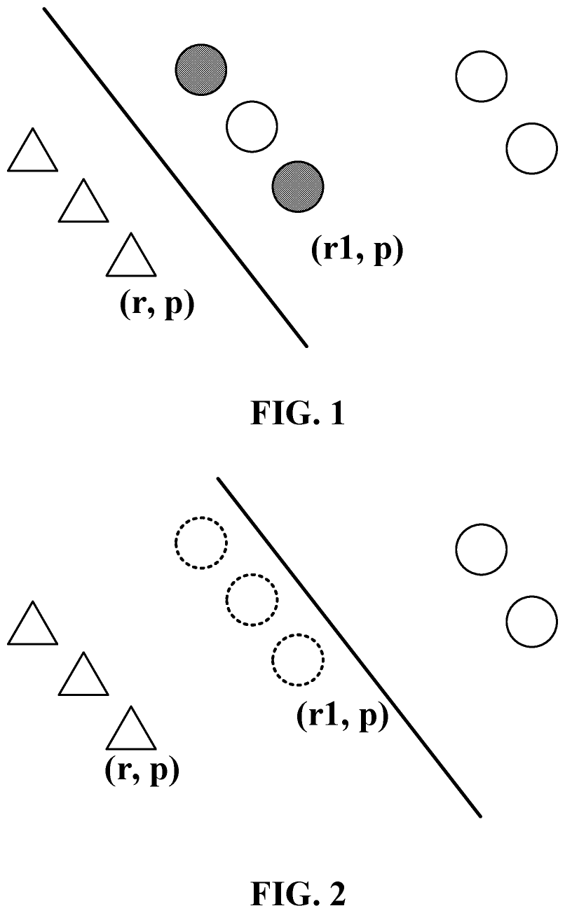

FIG. 1 illustrates a process of generating negative samples for predicting macromolecule-macromolecule interaction. Referring to FIG. 1, triangular shapes denote positive samples, circular shapes denote negative samples. In FIG. 1, shaded circular shapes denote negative samples that are similar to the positive samples. For example, a positive sample is denoted as (r, p), the similar negative sample may be denoted as (r1, p), wherein r1 is similar to r. Ideally, the positive sample data set for predicting macromolecule-macromolecule interaction (e.g., protein-RNA interaction) should not include the shaded circular shapes, and the negative sample data set should include these shaded circular shapes. The line between the triangular shapes and the shaded circular shapes indicates classification of samples into the positive sample data set or the negative sample data set.

FIG. 2 illustrates a process of generating negative samples for predicting macromolecule-macromolecule interaction. FIG. 2 illustrates a typical results using a method that randomly generates negative samples. Referring to FIG. 2, in a method that randomly generates negative samples, it is relatively easy to identify negative samples that are not closely similar (denoted by circular shapes with solid lines) to the positive samples. However, it is very difficult to identify negative samples that are similar (circular shapes with dotted lines, e.g., (r1, p)) to the positive samples. A machine learning classifier in macromolecule-macromolecule interaction (e.g., protein-RNA interaction) prediction may erroneously classify them into the positive sample data set, as indicated by the line between the circular shapes with dotted lines and the circular shapes with solid lines.

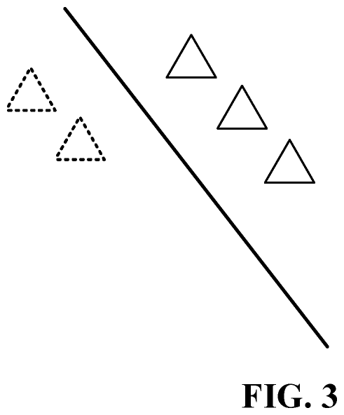

The inventors of the present disclosure discover that a degree of similarity in determining negative samples is another factor that could impact the accuracy of classification. FIG. 3 illustrates a process of generating negative samples for predicting macromolecule-macromolecule interaction. Referring to FIG. 3, triangular shapes with dotted lines denote sample points that are highly similar to the positive samples (triangular shapes with solid lines). These samples are in fact positive samples, but are erroneously identified as negative samples because a high degree of similarity is used in generating negative samples, resulting in misclassification of these sample points into the negative sample data set, as indicated by the line between the triangular shapes with dotted lines and the triangular shapes with solid lines.

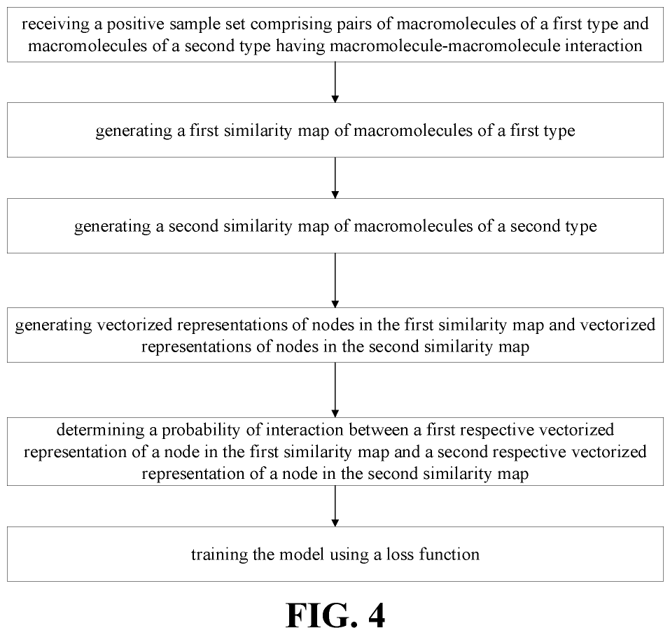

FIG. 4 illustrates a process of training a model for generating a negative sample set for predicting macromolecule-macromolecule interaction in some embodiments according to the present disclosure. Referring to FIG. 4, the present method in some embodiments includes generating a first similarity map of macromolecules of a first type (e.g., RNA molecules); and generating a second similarity map of macromolecules of a second type (e.g., protein molecules). In some embodiments, the first similarity map includes similarities among a plurality of pairs (e.g., all pairs) of the macromolecules of the first type; and the second similarity map includes similarities among a plurality of pairs (e.g., all pairs) of the macromolecules of the second type.

Various appropriate methods may be used for determining the similarity between pairs of macromolecules. Examples of appropriate methods for determining the similarity include edit distance comparison methods, token based comparison methods, and sequence based comparison methods. Edit distance comparison methods determine the number of operations required to transform a first macromolecular to a second macromolecular. The greater the number of operations required, the lower the similarity between the macromolecules. Specific examples of edit distance comparison methods include the Hamming distance method, the Levenshtein distance method, and the Jaro-Winkler method. In some embodiments, the similarity is calculated by a distance between a respective pair of macromolecules. In one example, a respective similarity between a respective pair of the macromolecules of the first type is expressed as:

$$sim 1 ( {m}_{1 - 1} , {m}_{1 - 2} ) = 1 - d 1 ( {m}_{1 - 1} , {m}_{1 - 2} ) ;$$

wherein (m₁₋₁, m₁₋₂) stands for the respective pair of the macromolecules of the first type, sim1 stands for the respective similarity between the respective pair of the macromolecules of the first type, and d1 stands for a distance between the respective pair of the macromolecules of the first type.

Optionally, the distance d1 is expressed as:

$$d 1 ( {m}_{1 - 1} , {m}_{1 - 2} ) = \frac{lev ( {m}_{1 - 1} , {m}_{1 - 2} )}{max ( len ( {m}_{1 - 1} ) , len ( {m}_{1 - 2} ) )} ;$$

wherein lev(m₁₋₁, m₁₋₂) stands for an edit distance between the respective pair of the macromolecules of the first type, len(m₁₋₁) stands for a length of a first macromolecule of the first type in the respective pair, and len(m₁₋₂) stands for a length of a second macromolecule of the first type in the respective pair.

In another example, a respective similarity between a respective pair of the macromolecules of the second type is expressed as:

$$sim 2 ( {m}_{2 - 1} , {m}_{2 - 2} ) = 1 - d 2 ( {m}_{2 - 1} , {m}_{2 - 2} ) ;$$

wherein (m₂₋₁, m₂₋₂) stands for the respective pair of the macromolecules of the second type, sim2 stands for the respective similarity between the respective pair of the macromolecules of the second type, and d2 stands for a distance between the respective pair of the macromolecules of the second type.

Optionally, the distance d2 is expressed as:

$$d 2 ( {m}_{2 - 1} , {m}_{2 - 2} ) = \frac{lev ( {m}_{2 - 1} , {m}_{2 - 2} )}{max ( len ( {m}_{2 - 1} ) , len ( {m}_{2 - 2} ) )} ;$$

wherein lev(m₂₋₁, m₂₋₂) stands for an edit distance between the respective pair of the macromolecules of the second type, len(m₂₋₁) stands for a length of a first macromolecule of the second type in the respective pair, and len(m₂₋₂) stands for a length of a second macromolecule of the second type in the respective pair.

In some embodiments, a node in the first similarity map represents a respective sequence of a respective macromolecule of the first type, an edge in the first similarity map connecting two adjacent nodes represents a distance between the respective pair of the macromolecules of the first type, and a weight of the edge represents the respective similarity between the two adjacent nodes. A node in the second similarity map represents a respective sequence of a respective macromolecule of the second type, an edge in the second similarity map connecting two adjacent nodes represents a distance between the respective pair of the macromolecules of the second type, and a weight of the edge represents the respective similarity between the two adjacent nodes.

Referring to FIG. 4, the present method in some embodiments further includes generating vectorized representations of nodes in the first similarity map; and generating vectorized representations of nodes in the second similarity map. In one specific example, the vectorized representations of nodes may be generated using a graph neural network GNN.

In some embodiments, the first similarity map includes N1 number of nodes, {eᵢ, i=1, . . . , N1}; and M1 number of edges, {rⱼ, j=1, . . . , M}. In one example, a respective vectorized representation of a respective node in the first similarity map is expressed as:

$${h}^{t 1 + 1} ( {e}_{i} ) = σ ( {W}_{p} \times {h}^{t 1} ( {e}_{i} ) + {\sum }_{{e}_{k} ∈ N ( {e}_{i} )} {W}_{ph} \times {h}^{t 1} ( {e}_{k} ) ) ;$$

wherein eᵢ stands for a respective node in the first similarity map; h^{t1}(eᵢ) stands for a respective vectorized representation of the respective node eᵢ prior to a t1-th step reiteration; h^{t1+1}(eᵢ) stands for an updated respective vectorized representation of the respective node eᵢ subsequent to the t1-th step reiteration; σ stands for a leaky relu activation function; N(eᵢ) stands for a set of nodes neighboring the respective node eᵢ; and Wₚ, Wₚₕ stand for parameters of a graph neural network for generating the vectorized representation. Optionally, the present method randomly initializing the parameters of the graph neural network and an initial respective vectorized representation h⁰(eᵢ) of the respective node eᵢ. Optionally, a maximum value of t1 may be used. In one example, the maximum value for t1 is a positive integer, e.g., 10.

In one example, the vectorized representations of nodes in the first similarity map are denoted by drᵢ.

In one example, the macromolecules of the first type is protein, the first similarity map includes similarities among a plurality of pairs of proteins; and the respective vectorized representation is calculated for the first similarity map including similarities among a plurality of pairs of proteins. In another example, the macromolecules of the first type is a RNA molecule, the first similarity map includes similarities among a plurality of pairs of RNA molecules; and the respective vectorized representation is calculated for the first similarity map including similarities among a plurality of pairs of RNA molecules. Optionally, the first similarity map is a similarity map for RNA molecules.

In some embodiments, the second similarity map includes N2 number of nodes, {e′ᵢ, i=1, . . . , N2}; and M2 number of edges, {r′ⱼ, j=1, . . . , M2}. In one example, a respective vectorized representation of a respective node in the second similarity map is expressed as:

$${h}^{t 2 + 1} ( {e}_{i}^{′} ) = σ ( {\sum }_{{e}_{k}^{′} ∈ N ( {e}_{i}^{′} ) ⋃ {e}_{i}^{′}} {α}_{i , k}^{t 2 + 1} {W}_{p}^{t + 1} \times {h}^{t 2} ( {e}_{k}^{′} ) ) ; \\ {α}_{i , k}^{t 2 + 1} = softmax ( 〈 {h}^{t 2} ( {e}_{i}^{′} ) , {h}^{t 2} ( {e}_{k}^{′} ) 〉 ) ;$$

wherein e′ᵢ stands for a respective node in the second similarity map; h^{t2}(e′ᵢ) stands for a respective vectorized representation of the respective node e′ᵢ prior to a t2-th step reiteration; h^{t2+1}(e′ᵢ) stands for an updated respective vectorized representation of the respective node e′ᵢ subsequent to the t2-th step reiteration; σ stands for a leaky relu activation function; N(e′ᵢ) stands for a set of nodes neighboring the respective node e′ᵢ; <h^{t2}(eᵢ′), h^{t2}(eₖ′)> stands for an inner product of h^{t2}(eᵢ′) and h^{t2}(eₖ′); Wₚ^{t2+1} stands for a parameter of a graph neural network for generating the vectorized representation; and α_{i,k}^{t2+1} stands for attention weights representing a link strength between node eᵢ′ and node eₖ′. Optionally, the present method randomly initializing the parameters of the graph neural network and an initial respective vectorized representation h⁰(e′ᵢ) of the respective node e′ᵢ. Optionally, a maximum value of t2 may be used. In one example, the maximum value for t2 is a positive integer, e.g., 6.

In one example, the vectorized representations of nodes in the second similarity map are denoted by dpⱼ.

In one example, the macromolecules of the second type is a RNA molecule, the second similarity map includes similarities among a plurality of pairs of RNA molecules; and the respective vectorized representation is calculated for the second similarity map including similarities among a plurality of pairs of RNA molecules. In another example, the macromolecules of the second type is protein, the second similarity map includes similarities among a plurality of pairs of proteins; and the respective vectorized representation is calculated for the second similarity map including similarities among a plurality of pairs of proteins. Optionally, the second similarity map is a similarity map for protein molecules.

Referring to FIG. 4, the present method in some embodiments further includes determining a probability of interaction between a first respective vectorized representation of a node in the first similarity map and a second respective vectorized representation of a node in the second similarity map by:

$$P ( 1 \vert dm {1}_{i} , dm {2}_{j} ) = \frac{1}{1 + exp ( - θ \cdot [ dm {1}_{i} , dm {2}_{j} , dm {1}_{i} - dm {2}_{j} , dm {1}_{i} ⊗ dm {2}_{j} ] )} ;$$

wherein dm1ᵢ stands for vectorized representations of nodes in the first similarity map; dm2ⱼ stands for vectorized representations of nodes in the second similarity map; p(1|dm1ᵢ, dm2ᵢ) stands for a probability of interaction between a first respective vectorized representation of a node in the first similarity map and a second respective vectorized representation of a node in the second similarity map; [,] stands for stitching between elements separated by the comma (e.g., [dm1ᵢ, dm2ⱼ, dm1ᵢ−dm2ⱼ, dm1ᵢ⊗dm2ⱼ] stands for stitching between dm1ᵢ, dm2ⱼ, dm1ᵢ−dm2ⱼ, and dm1ᵢ⊗dm2ⱼ); ⊗ stands for product of two vectors; and θ stands for a parameter that is tunable (e.g., a parameter that is tunable by a training process).

In some embodiments, the present method further includes training a model using a positive sample set {(m1ᵢ, m2ᵢ), i=1, . . . , K}, wherein m1 stands for a macromolecule of a first type and m2 stands for a macromolecule of a second type. Various appropriate methods may be used for training the model. In one example, the present method includes training the model using stochastic gradient descent to minimize a loss function L:

$$L = - {\sum }_{i = 1}^{K} log p ( 1 \vert m {1}_{i} , m {2}_{i} ) .$$

By minimizing the loss function L, the model can be fine-tuned and parameters of the model can be optimized to better detect interaction between macromolecules of the first type and macromolecules of the second type. Because the input to the model includes the first similarity map and the second similarity map, machine learning can be performed using these similarity maps. Examples of parameters to be optimized by minimizing the loss function L include Wₚ, Wₚₕ, and θ.

In another aspect, the present disclosure provides a neural network model for predicting macromolecule-macromolecule interaction, trained by the method of training a model for generating a negative sample set for predicting macromolecule-macromolecule interaction described herein.

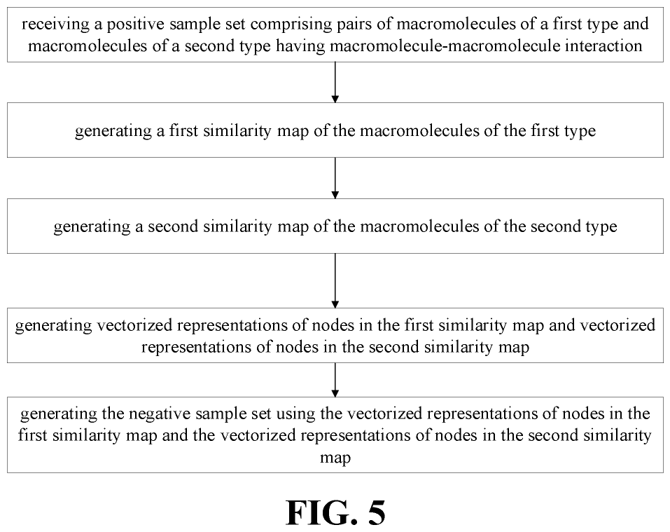

In another aspect, the present disclosure provides a method of generating a negative sample set for predicting macromolecule-macromolecule interaction. FIG. 5 illustrates a process of generating a negative sample set for predicting macromolecule-macromolecule interaction in some embodiments according to the present disclosure. Referring to FIG. 5, the method in some embodiments includes receiving a positive sample set comprising pairs of macromolecules of a first type and macromolecules of a second type having macromolecule-macromolecule interaction; generating a first similarity map of the macromolecules of the first type; generating a second similarity map of the macromolecules of the second type; generating vectorized representations of nodes in the first similarity map and vectorized representations of nodes in the second similarity map; and generating the negative sample set using the vectorized representations of nodes in the first similarity map and the vectorized representations of nodes in the second similarity map. The vectorized representations of nodes, the first similarity map, and the second similarity map may be generated and stored in any appropriate manner. In one example, the vectorized representations of nodes, the first similarity map, and the second similarity map are generated each time the method is executed to generate a negative sample set, e.g., ab initio. In another example, the vectorized representations of nodes, the first similarity map, and the second similarity map are generated during a process of training the model, and stored in a memory for later use in generating one or more negative sample sets.

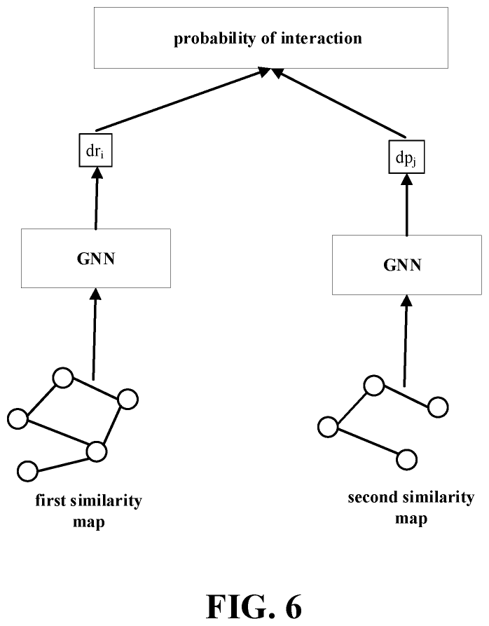

FIG. 6 illustrate a specific example of generating a negative sample set for predicting macromolecule-macromolecule interaction in some embodiments according to the present disclosure. Referring to FIG. 6, a node in the first similarity map represents a respective sequence of a respective macromolecule of the first type, and an edge in the first similarity map connecting two adjacent nodes represents a distance between the respective pair of the macromolecules of the first type, and weight of the edge represents the respective similarity between the two adjacent nodes. A node in the second similarity map represents a respective sequence of a respective macromolecule of the second type, and an edge in the second similarity map connecting two adjacent nodes represents a distance between the respective pair of the macromolecules of the second type, and a weight of the edge represents the respective similarity between the two adjacent nodes. As shown in FIG. 6, in one specific example, the vectorized representations of nodes may be generated using a graph neural network GNN.

Referring to FIG. 5 and FIG. 6, in some embodiments, the method of generating the negative sample set includes receiving a positive sample set {(m1ᵢ, m2ᵢ), i=1, . . . , K}, and generating vectorized representations drᵢ of nodes in the first similarity map and vectorized representations dpⱼ of nodes in the second similarity map.

In some embodiments, generating the negative sample set further includes, with respect to m2ᵢ, calculating similarities between m1ᵢ and m1ⱼ (j=1, . . . , K, and j≠i). Various appropriate algorithms may be used for calculating similarities. Examples of appropriate algorithms include Match, Shingliing, SimHash, Random Projection, and SpotSig. In one example, the similarities between m1ᵢ and m1ⱼ (j=1, . . . , K, and j≠i) is calculated by:

$$S ( {dr}_{j} , {dr}_{i} ) = \frac{〈 {dr}_{j} , {dr}_{i} 〉}{\sqrt{〈 {dr}_{j} , {dr}_{j} 〉} \times \sqrt{〈 {dr}_{i} , {dr}_{i} 〉}} .$$

In some embodiments, generating the negative sample set further includes sorting m1ⱼ (j=1, . . . , K, and j≠i) (e.g., in descending order or ascending order) based on the similarities between m1ᵢ and m1ⱼ to obtain a subset; determining a probability of interaction between m2ᵢ and each sample in the subset; and generating the respective intermediate set based on the probability of interaction. Various appropriate algorithms may be used to determine probability of interaction between two samples. Examples of appropriate algorithms for determining probability of interaction between two macromolecules include sequence-based methods, structure-based methods, function-based methods, co-evolutionary profile-based methods, or any combination thereof. The inventors of the present disclosure discover a unique vector-based method for determining probability of interaction. In some embodiments, probability of interaction between m2ᵢ and each sample in the subset may be determined by:

$$P ( 1 \vert {dr}_{j} , {dp}_{i} ) = \frac{1}{1 + exp ( - θ \cdot [ {dr}_{j} , {dp}_{i} , {dr}_{j} - {dp}_{i} , {dr}_{j} ⊗ {dp}_{i} ] )} ;$$

wherein drⱼ stands for vectorized representations of nodes in the subset; dpᵢ stands for vectorized representations of m2ᵢ; P(1|drⱼ, dpᵢ) stands for a probability of interaction between m2ᵢ and each sample in the subset; [,] stands for stitching between elements separated by the comma (e.g., [drⱼ, dpᵢ, drⱼ−dpᵢ, drⱼ⊗dpᵢ] stands for stitching between elements drⱼ, dpᵢ, drⱼ−dpᵢ, and drⱼ⊗dpᵢ); ⊗ stands for product of two vectors; and θ stands for a parameter that is tunable (e.g., a parameter that is tunable by a training process).

In some embodiments, generating the negative sample set further includes generating a plurality of intermediate sets; and sampling L number of negative samples from a respective intermediate set of the plurality of intermediate sets. Optionally, the negative sample set includes negative samples sampled from the plurality of intermediate sets; L is an integer equal to or greater than 1, e.g., 1, 2, 3, 4, 5, or 6. Optionally, the negative sample set consists of a single negative sample, accordingly generating the negative sample set includes generating a single intermediate set; and sampling a single negative sample from the single intermediate sample set.

In some embodiments, the positive sample set is represented by {(m1ᵢ, m2ᵢ), i=1, . . . , K}, wherein m1ᵢ stands for an i-th macromolecule of the first type and m2ᵢ stands for an i-th macromolecule of the second type. Optionally, generating the respective intermediate set of the plurality of intermediate sets includes determining a probability of interaction between m2ᵢ and each sample in m1ᵢ; and generating the respective intermediate set based on the probability of interaction.

In one example, generating the negative sample set further includes placing (m1ⱼ, 1−P(1|drⱼ, dpᵢ)) into an intermediate set when P(1|drⱼ, dpᵢ) is less than a threshold value, e.g., 0.4, 0.45, 0.5, 0.55, or 0.6. In one example, the threshold value is 0.5. If the intermediate set is an empty set, it indicates that a given positive sample cannot be used to generate a negative sample.

In some embodiments, when the intermediate set is not an empty, generating the negative sample set further includes sampling L number of negative samples from the intermediate set based on probability {pₖ, k=1, . . . , |T|}, wherein

$${p}_{k} = \frac{1 - P ( 1 \vert {dr}_{j} , {dp}_{i} )}{{\sum }_{l = 1}^{\vert T \vert} ( 1 - P ( 1 \vert {dr}_{j} , {dp}_{i} ) )} ; and$$

|T| stands for a number of elements in the intermediate set, and (m1ⱼ, 1−P(1|drⱼ, dpᵢ)) stands for a k-th element in the intermediate set.

Optionally, the macromolecules of the first type comprise RNA molecules and macromolecules of the second type comprise protein molecules.

Optionally, the macromolecules of the first type comprise protein molecules and macromolecules of the second type comprise RNA molecules.

Referring to FIG. 5 again, the method in some embodiments includes generating a first similarity map of the macromolecules of the first type. As discussed above, in some embodiments, a respective similarity between a respective pair of the macromolecules of the first type is expressed as:

$$sim 1 ( {m}_{1 - 1} , {m}_{1 - 2} ) = 1 - d 1 ( {m}_{1 - 1} , {m}_{1 - 2} ) ;$$

wherein (m₁₋₁, m₁₋₂) stands for the respective pair of the macromolecules of the first type, sim1 stands for the respective similarity between the respective pair of the macromolecules of the first type, and d1 stands for a distance between the respective pair of the macromolecules of the first type.

Optionally, the distance d1 is expressed as:

$$d 1 ( {m}_{1 - 1} , {m}_{1 - 2} ) = \frac{lev ( {m}_{1 - 1} , {m}_{1 - 2} )}{max ( len ( {m}_{1 - 1} ) , len ( {m}_{1 - 2} ) )} ;$$

wherein lev(m₁₋₁, m₁₋₂) stands for an edit distance between the respective pair of the macromolecules of the first type, len(m₁₋₁) stands for a length of a first macromolecule of the first type in the respective pair, and len(m₁₋₂) stands for a length of a second macromolecule of the first type in the respective pair.

Referring to FIG. 5 again, the method in some embodiments includes generating a second similarity map of the macromolecules of the second type; As discussed above, in some embodiments, a respective similarity between a respective pair of the macromolecules of the second type is expressed as:

$$si m 2 ( {m}_{2 - 1} , {m}_{2 - 2} ) = 1 - d 2 ( {m}_{2 - 1} , {m}_{2 - 2} ) ;$$

wherein (m₂₋₁, m₂₋₂) stands for the respective pair of the macromolecules of the second type, sim2 stands for the respective similarity between the respective pair of the macromolecules of the second type, and d2 stands for a distance between the respective pair of the macromolecules of the second type.

Optionally, the distance d2 is expressed as:

$$d 2 ( {m}_{2 - 1} , {m}_{2 - 2} ) = \frac{lev ( {m}_{2 - 1} , {m}_{2 - 2} )}{max ( len ( {m}_{2 - 1} ) , len ( {m}_{2 - 2} ) )} ;$$

wherein lev(m₂₋₁, m₂₋₂) stands for an edit distance between the respective pair of the macromolecules of the second type, len(m₂₋₁) stands for a length of a first macromolecule of the second type in the respective pair, and len(m₂₋₂) stands for a length of a second macromolecule of the second type in the respective pair.

Referring to FIG. 5 again, the method in some embodiments includes generating vectorized representations of nodes in the first similarity map and vectorized representations of nodes in the second similarity map. As discussed above, in some embodiments, the first similarity map includes N1 number of nodes, {eᵢ, i=1, . . . , N1}; and M1 number of edges, {rⱼ, j=1, . . . , M}. In one example, a respective vectorized representation of a respective node in the first similarity map is expressed as:

$${h}^{t 1 + 1} ( {e}_{i} ) = σ ( {W}_{p} \times {h}^{t 1} ( {e}_{i} ) + {\sum }_{{e}_{k} ∈ N ( {e}_{i} )} {W}_{p h} \times {h}^{t 1} ( {e}_{k} ) ) ;$$

wherein eᵢ stands for a respective node in the first similarity map; h^{t1}(eᵢ) stands for a respective vectorized representation of the respective node eᵢ prior to a t1-th step reiteration; h^{t1+1}(eᵢ) stands for an updated respective vectorized representation of the respective node eᵢ subsequent to the t1-th step reiteration; σ stands for a leaky relu activation function; N(eᵢ) stands for a set of nodes neighboring the respective node eᵢ; and Wₚ, Wₚₕ stand for parameters of a graph neural network for generating the vectorized representation. Optionally, the present method randomly initializing the parameters of the graph neural network and an initial respective vectorized representation h⁰(eᵢ) of the respective node eᵢ. Optionally, a maximum value of t1 may be used. In one example, the maximum value for t1 is a positive integer, e.g., 10.

In one example, the vectorized representations of nodes in the first similarity map are denoted by drᵢ.

In some embodiments, the second similarity map includes N2 number of nodes, {e′ᵢ, i=1, . . . , N2}; and M2 number of edges, {r′ⱼ, j=1, . . . , M2}. In one example, a respective vectorized representation of a respective node in the second similarity map is expressed as:

$${h}^{t 2 + 1} ( {e}_{i}^{′} ) = σ ( {\sum }_{{e}_{k}^{′} ∈ N ( {e}_{i}^{′} ) ⋃ {e}_{i}^{′}} {α}_{i , k}^{t 2 + 1} {W}_{p}^{t + 1} \times {h}^{t 2} ( {e}_{k}^{′} ) ) ; {α}_{i , k}^{t 2 + 1} = softmax ( 〈 {h}^{t2} ( {e}_{i}^{′} ) , {h}^{t 2} ( {e}_{k}^{′} ) 〉 ) ;$$

wherein e′ᵢ stands for a respective node in the second similarity map; h^{t2}(e′ᵢ) stands for a respective vectorized representation of the respective node e′ᵢ prior to a t2-th step reiteration; h^{t2+1}(e′ᵢ) stands for an updated respective vectorized representation of the respective node e′ᵢ subsequent to the t2-th step reiteration; σ stands for a leaky relu activation function; N(e′ᵢ) stands for a set of nodes neighboring the respective node e′ᵢ; <h^{t2}(eᵢ′), h^{t2}(eₖ′)> stands for an inner product of h^{t2}(eᵢ′) and h^{t2}(eₖ′); Wₚ^{t2+1} stands for a parameter of a graph neural network for generating the vectorized representation; and α_{i,k}^{t2+1} stands for attention weights representing a link strength between node eᵢ′ and node eₖ′. Optionally, the present method randomly initializing the parameters of the graph neural network and an initial respective vectorized representation h⁰(e′ᵢ) of the respective node e′ᵢ. Optionally, a maximum value of t2 may be used. In one example, the maximum value for t2 is a positive integer, e.g., 6.

In one example, the vectorized representations of nodes in the second similarity map are denoted by dpⱼ.

In another aspect, the present disclosure provides a method of predicting macromolecule-macromolecule interaction using a positive sample set and the negative sample set generated by a method described in the present disclosure.

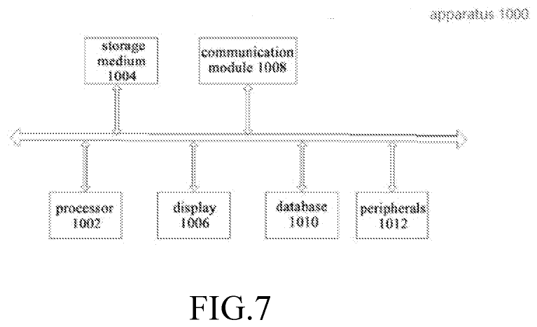

In another aspect, the present disclosure provides an apparatus. FIG. 7 is a schematic diagram illustrating an apparatus in some embodiments according to the present disclosure. Referring to FIG. 7, the apparatus 1000 may include any appropriate type of TV, such as a plasma TV, a liquid crystal display (LCD) TV, a touch screen TV, a projection TV, a non-smart TV, a smart TV, etc. The apparatus 1000 may also include other computing systems, such as a personal computer (PC), a tablet or mobile computer, or a smart phone, etc. In addition, the apparatus 1000 may be any appropriate content-presentation device capable of presenting any appropriate content. Users may interact with the apparatus 1000 to perform other activities of interest.

As shown in FIG. 7, the apparatus 1000 may include a processor 1002, a storage medium 1004, a display 1006, a communication module 1008, a database 1010 and peripherals 1012. Certain devices may be omitted, and other devices may be included to better describe the relevant embodiments.

The processor 1002 may include any appropriate processor or processors. Further, the processor 1002 may include multiple cores for multi-thread or parallel processing. The processor 1002 may execute sequences of computer program instructions to perform various processes. The storage medium 1004 may include memory modules, such as ROM, RAM, flash memory modules, and mass storages, such as CD-ROM and hard disk, etc. The storage medium 1004 may store computer programs for implementing various processes when the computer programs are executed by the processor 1002. For example, the storage medium 1004 may store computer programs for implementing various algorithms when the computer programs are executed by the processor 1002.

Further, the communication module 1008 may include certain network interface devices for establishing connections through communication networks, such as TV cable network, wireless network, internet, etc. The database 1010 may include one or more databases for storing certain data and for performing certain operations on the stored data, such as database searching.

The display 1006 may provide information to users. The display 1006 may include any appropriate type of computer display device or electronic apparatus display such as LCD or OLED based devices. The peripherals 112 may include various sensors and other I/O devices, such as keyboard and mouse.

All or some of steps of the method, functional modules/units in the system and the device disclosed above may be implemented as software, firmware, hardware, or suitable combinations thereof. In a hardware implementation, a division among functional modules/units mentioned in the above description does not necessarily correspond to the division among physical components. For example, one physical component may have a plurality of functions, or one function or step may be performed by several physical components in cooperation. Some or all of the physical components may be implemented as software executed by a processor, such as a central processing unit, a digital signal processor, or a microprocessor, or as hardware, or as an integrated circuit, such as an application specific integrated circuit. Such software may be distributed on a computer-readable storage medium, which may include a computer storage medium (or a non-transitory medium) and a communication medium (or a transitory medium). The term computer storage medium includes volatile and nonvolatile, removable and non-removable media implemented in any method or technology for storage of information, such as computer readable instructions, data structures, program modules or other data, as is well known to one of ordinary skill in the art. A computer storage medium includes, but is not limited to, RAM, ROM, EEPROM, flash memory or other memory technology, CD-ROM, Digital Versatile Disk (DVD) or other optical disk storage, magnetic cassette, magnetic tape, magnetic disk storage or other magnetic storage device, or any other medium which may be used to store desired information, and which may be accessed by a computer. In addition, a communication medium typically embodies computer readable instructions, data structures, program modules or other data in a modulated data signal such as a carrier wave or other transport mechanism, and includes any information delivery medium, as is well known to one of ordinary skill in the art.

The flowchart and block diagrams in the drawings illustrate architecture, functionality, and operation of possible implementations of a device, a method and a computer program product according to various embodiments of the present disclosure. In this regard, each block in the flowchart or block diagrams may represent a module, program segment(s), or a portion of a code, which includes at least one executable instruction for implementing specified logical function(s). It should also be noted that, in some alternative implementations, functions noted in the blocks may occur out of the order noted in the drawings. For example, two blocks being successively connected may, in fact, be performed substantially concurrently, or the blocks may sometimes be performed in the reverse order, depending upon the functionality involved. It will also be noted that each block of the block diagrams and/or flowchart, and combinations of blocks in the block diagrams and/or flowchart, may be implemented by special purpose hardware-based systems that perform the specified functions or operations, or combinations of special purpose hardware and computer instructions.

In some embodiments, the apparatus includes one or more memory, and one or more processors, wherein the one or more memory and the one or more processors are connected with each other. In some embodiments, the one or more memory stores computer-executable instructions for controlling the one or more processors to receive a positive sample set comprising pairs of macromolecules of a first type and macromolecules of a second type having macromolecule-macromolecule interaction; generate a first similarity map of the macromolecules of the first type; generate a second similarity map of the macromolecules of the second type; generate vectorized representations of nodes in the first similarity map and vectorized representations of nodes in the second similarity map; and generate the negative sample set using the vectorized representations of nodes in the first similarity map and the vectorized representations of nodes in the second similarity map.

In some embodiments, the one or more memory stores computer-executable instructions for controlling the one or more processors to receive a positive sample set comprising pairs of macromolecules of a first type and macromolecules of a second type having macromolecule-macromolecule interaction; generate a first similarity map of macromolecules of a first type; generate a second similarity map of macromolecules of a second type; generate vectorized representations of nodes in the first similarity map and vectorized representations of nodes in the second similarity map; determine a probability of interaction between a first respective vectorized representation of a node in the first similarity map and a second respective vectorized representation of a node in the second similarity map; and train the model using a loss function.

In another aspect, the present disclosure provides a computer-program product including a non-transitory tangible computer-readable medium having computer-readable instructions thereon. In some embodiments, the computer-readable instructions being executable by a processor to cause the processor to perform receiving a positive sample set comprising pairs of macromolecules of a first type and macromolecules of a second type having macromolecule-macromolecule interaction; generating a first similarity map of the macromolecules of the first type; generating a second similarity map of the macromolecules of the second type; generating vectorized representations of nodes in the first similarity map and vectorized representations of nodes in the second similarity map; and generating the negative sample set using the vectorized representations of nodes in the first similarity map and the vectorized representations of nodes in the second similarity map.

In some embodiments, the computer-readable instructions being executable by a processor to cause the processor to perform receiving a positive sample set comprising pairs of macromolecules of a first type and macromolecules of a second type having macromolecule-macromolecule interaction; generating a first similarity map of macromolecules of a first type; generating a second similarity map of macromolecules of a second type; generating vectorized representations of nodes in the first similarity map and vectorized representations of nodes in the second similarity map; determining a probability of interaction between a first respective vectorized representation of a node in the first similarity map and a second respective vectorized representation of a node in the second similarity map; and training the model using a loss function.

Various illustrative neural networks, layers, units, channels, blocks, and other operations described in connection with the configurations disclosed herein may be implemented as electronic hardware, computer software, or combinations of both. Such neural networks, layers, units, channels, blocks, and other operations may be implemented or performed with a general purpose processor, a digital signal processor (DSP), an ASIC or ASSP, an FPGA or other programmable logic device, discrete gate or transistor logic, discrete hardware components, or any combination thereof designed to produce the configuration as disclosed herein. For example, such a configuration may be implemented at least in part as a hard-wired circuit, as a circuit configuration fabricated into an application-specific integrated circuit, or as a firmware program loaded into non-volatile storage or a software program loaded from or into a data storage medium as machine-readable code, such code being instructions executable by an array of logic elements such as a general purpose processor or other digital signal processing unit. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. A software module may reside in a non-transitory storage medium such as RAM (random-access memory), ROM (read-only memory), nonvolatile RAM (NVRAM) such as flash RAM, erasable programmable ROM (EPROM), electrically erasable programmable ROM (EEPROM), registers, hard disk, a removable disk, or a CD-ROM; or in any other form of storage medium known in the art. An illustrative storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

The foregoing description of the embodiments of the invention has been presented for purposes of illustration and description. It is not intended to be exhaustive or to limit the invention to the precise form or to exemplary embodiments disclosed. Accordingly, the foregoing description should be regarded as illustrative rather than restrictive. Obviously, many modifications and variations will be apparent to practitioners skilled in this art. The embodiments are chosen and described in order to explain the principles of the invention and its best mode practical application, thereby to enable persons skilled in the art to understand the invention for various embodiments and with various modifications as are suited to the particular use or implementation contemplated. It is intended that the scope of the invention be defined by the claims appended hereto and their equivalents in which all terms are meant in their broadest reasonable sense unless otherwise indicated. Therefore, the term “the invention”, “the present invention” or the like does not necessarily limit the claim scope to a specific embodiment, and the reference to exemplary embodiments of the invention does not imply a limitation on the invention, and no such limitation is to be inferred. The invention is limited only by the spirit and scope of the appended claims. Moreover, these claims may refer to use “first”, “second”, etc. following with noun or element. Such terms should be understood as a nomenclature and should not be construed as giving the limitation on the number of the elements modified by such nomenclature unless specific number has been given. Any advantages and benefits described may not apply to all embodiments of the invention. It should be appreciated that variations may be made in the embodiments described by persons skilled in the art without departing from the scope of the present invention as defined by the following claims. Moreover, no element and component in the present disclosure is intended to be dedicated to the public regardless of whether the element or component is explicitly recited in the following claims.

1. A method of generating a negative sample set for predicting macromolecule-macromolecule interaction, comprising:
receiving a positive sample set comprising pairs of macromolecules of a first type and macromolecules of a second type having macromolecule-macromolecule interaction;
generating a first similarity map of the macromolecules of the first type;
generating a second similarity map of the macromolecules of the second type;
generating vectorized representations of nodes in the first similarity map and vectorized representations of nodes in the second similarity map; and
generating the negative sample set using the vectorized representations of nodes in the first similarity map and the vectorized representations of nodes in the second similarity map.

2. The method of claim 1, wherein the first similarity map or the second similarity map comprises nodes and edges connecting adjacent nodes, wherein a respective node represents a respective macromolecule, a respective edge represents a respective distance between a respective pair of the macromolecules, and a respective weight of the respective edge represents a respective similarity between the respective pair of the macromolecules.

3. The method of claim 1, further comprising generating a plurality of intermediate sets;
wherein the positive sample set is represented by {(m1ᵢ, m2ᵢ), i=1, . . . , K}, wherein m1ᵢ stands for an i-th macromolecule of the first type and m2ᵢ stands for an i-th macromolecule of the second type;
wherein generating the respective intermediate set of the plurality of intermediate sets comprises:
sorting m1ⱼ (j=1, . . . , K, and j≠i) based on similarities between m1ᵢ and m1ⱼ to obtain a subset of m1ⱼ (j=1, . . . , K, and j≠i);
determining a probability of interaction between m2ᵢ and each sample in the subset; and
generating the respective intermediate set based on the probability of interaction.

4. The method of claim 3, further comprising calculating similarities between m1ᵢ and m1ⱼ (j=1, . . . , K, and j≠i) by:
$$S ( d {r}_{j} , {dr}_{i} ) = \frac{< d {r}_{j} , {dr}_{i} >}{\sqrt{< d {r}_{j} , {dr}_{j} >} \times \sqrt{< d {r}_{i} , {dr}_{i} >}} ;$$
wherein drⱼ stands for vectorized representations of nodes in the subset; and drᵢ stands for a vectorized representation of m1ᵢ.

5. The method of claim 3, wherein the probability of interaction between m2ᵢ and each sample in the subset is determined by:
$$P ( 1 \vert {dr}_{j} , {dp}_{i} ) = \frac{1}{1 + exp ( - θ \cdot [ {dr}_{j} , {dp}_{i} , {dr}_{j} - d {p}_{i} , {dr}_{j} ⊗ d {p}_{i} ] )} ;$$
wherein drⱼ stands for vectorized representations of nodes in the subset; dpᵢ stands for vectorized representations of m2ᵢ; P(1|drⱼ, dpᵢ) stands for a probability of interaction between m2ᵢ and each sample in the subset; [,] stands for stitching between elements; ⊗ stands for product of two vectors; and θ stands for a parameter that is tunable.

6. The method of claim 4, further comprising placing (m1ⱼ, 1−P(1|drⱼ, dpᵢ)) into the respective intermediate set when P(1|drⱼ, dpᵢ) is less than a threshold value.

7. The method of claim 5, wherein sampling L number of negative samples from the respective intermediate set is performed based on probability {pₖ, k=1, . . . , |T|};
wherein
$${p}_{k} = \frac{1 - P ( 1 \vert {dr}_{j} , {dp}_{i} )}{{\sum }_{l = 1}^{\vert T \vert} ( 1 - P ( 1 \vert {dr}_{j} , {dp}_{i} ) )} ;$$

|T| stands for a number of elements in the respective intermediate set; and
(m1ⱼ, 1−P(1|drⱼ, dpᵢ)) stands for a k-th element in the intermediate set.

8. The method of claim 3, wherein generating the negative sample set comprises:
sampling L number of negative samples from a respective intermediate set of the plurality of intermediate sets;
wherein the negative sample set comprises negative samples sampled from the plurality of intermediate sets; and
L is an integer equal to or greater than 1.

9. (canceled)

10. A method of predicting macromolecule-macromolecule interaction using the positive sample set and the negative sample set generated by the method of claim 1.

11. A method of training a model for generating a negative sample set for predicting macromolecule-macromolecule interaction, comprising:
receiving a positive sample set comprising pairs of macromolecules of a first type and macromolecules of a second type having macromolecule-macromolecule interaction;
generating a first similarity map of macromolecules of a first type;
generating a second similarity map of macromolecules of a second type;
generating vectorized representations of nodes in the first similarity map and vectorized representations of nodes in the second similarity map;
determining a probability of interaction between a first respective vectorized representation of a node in the first similarity map and a second respective vectorized representation of a node in the second similarity map; and
training the model at least partially based on the probability of interaction.

12. The method of claim 11, wherein the probability of interaction is determined by:
$$P ( 1 \vert dm {1}_{i} , dm {2}_{j} ) = \frac{1}{1 + exp ( - θ \cdot [ dm {1}_{i} , dm {2}_{j} , dm {1}_{i} - d m {2}_{j} , dm {1}_{i} ⊗ dm {2}_{j} ] )} ;$$
wherein dm1ᵢ stands for vectorized representations of nodes in the first similarity map; dm2ᵢ stands for vectorized representations of nodes in the second similarity map; p(1|dm1ᵢ, dm2ᵢ) stands for a probability of interaction between a first respective vectorized representation of a node in the first similarity map and a second respective vectorized representation of a node in the second similarity map; [,] stands for stitching between elements; and ⊗ stands for product of two vectors; and θ stands for a parameter that is tunable.

13. The method of claim 11, wherein the first similarity map or the second similarity map comprises nodes and edges connecting adjacent nodes, wherein a respective node represents a respective macromolecule, a respective edge represents a respective distance between a respective pair of the macromolecules, and a respective weight of the respective edge represents a respective similarity between the respective pair of the macromolecules.

14. The method of claim 11, wherein a respective similarity between a respective pair of the macromolecules of the first type is expressed as:
$$si 1 ( {m}_{1 - 1} , {m}_{1 - 2} ) = 1 - d 1 ( {m}_{1 - 1} , {m}_{1 - 2} ) ;$$
wherein (m₁₋₁, m₁₋₂) stands for the respective pair of the macromolecules of the first type, sim1 stands for the respective similarity between the respective pair of the macromolecules of the first type, and d1 stands for a distance between the respective pair of the macromolecules of the first type.

15. The method of claim 14, wherein d1 is expressed as:
$$d 1 ( {m}_{1 - 1} , {m}_{1 - 2} ) = \frac{l e v ( {m}_{1 - 1} , {m}_{1 - 2} )}{max ( l e n ( {m}_{1 - 1} ) , le n ( {m}_{1 - 2} ) )} ;$$
wherein lev(m₁₋₁, m₁₋₂) stands for an edit distance between the respective pair of the macromolecules of the first type, len(m₁₋₁) stands for a length of a first macromolecule of the first type in the respective pair, and len(m₁₋₂) stands for a length of a second macromolecule of the first type in the respective pair.

16. The method of claim 11, wherein a respective similarity between a respective pair of the macromolecules of the second type is expressed as:
$$sim 2 ( {m}_{2 - 1} , {m}_{2 - 2} ) = 1 - d 2 ( {m}_{2 - 1} , {m}_{2 - 2} ) ;$$
wherein (m₂₋₁, m₂₋₂) stands for the respective pair of the macromolecules of the second type, sim2 stands for the respective similarity between the respective pair of the macromolecules of the second type, and d2 stands for a distance between the respective pair of the macromolecules of the second type.

17. The method of claim 16, wherein d2 is expressed as:
$$d 2 ( {m}_{2 - 1} , {m}_{2 - 2} ) = \frac{l e v ( {m}_{2 - 1} , {m}_{2 - 2} )}{max ( l e n ( {m}_{2 - 1} ) , le n ( {m}_{2 - 2} ) )} ;$$
wherein lev(m₂₋₁, m₂₋₂) stands for an edit distance between the respective pair of the macromolecules of the second type, len(m₂₋₁) stands for a length of a first macromolecule of the second type in the respective pair, and len(m₂₋₂) stands for a length of a second macromolecule of the second type in the respective pair.

18. The method of claim 11, wherein the first similarity map includes N1 number of nodes, {eᵢ, i=1, . . . , N1}; and M1 number of edges, {rⱼ, j=1, . . . , M};
a respective vectorized representation of a respective node in the first similarity map is expressed as:
$${h}^{t 1 + 1} ( {e}_{i} ) = σ ( {W}_{p} \times {h}^{t 1} ( {e}_{i} ) + {\sum }_{{e}_{k} ∈ N ( {e}_{i} )} {W}_{p h} \times {h}^{t 1} ( {e}_{k} ) ) ;$$
wherein eᵢ stands for a respective node in the first similarity map; h^{t1}(eᵢ) stands for a respective vectorized representation of the respective node eᵢ prior to a t1-th step reiteration; h^{t1+1}(eᵢ) stands for an updated respective vectorized representation of the respective node eᵢ subsequent to the t1-th step reiteration; σ stands for a leaky relu activation function; N(eᵢ) stands for a set of nodes neighboring the respective node eᵢ; and Wₚ, Wₚₕ stand for parameters of a graph neural network for generating the vectorized representation.

19. The method of claim 11, wherein the second similarity map includes N2 number of nodes, {e′ᵢ, i=1, . . . , N2}; and M2 number of edges, {r′ⱼ, j=1, . . . , M2};
a respective vectorized representation of a respective node in the second similarity map is expressed as:
$$\begin{matrix}{h}^{t 2 + 1} ( {e}_{i}^{′} ) = σ ( {\sum }_{{e}_{k}^{′} ∈ N ( {e}_{i}^{′} ) ⋃ {e}_{i}} {α}_{i , k}^{t 2 + 1} {W}_{p}^{t + 1} \times {h}^{t 2} ( {e}_{k}^{′} ) ) ; \\ {α}_{i , k}^{t 2 + 1} = softmax ( 〈 {h}^{t 2} ( {e}_{i}^{′} ) , {h}^{t 2} ( {e}_{k}^{′} ) 〉 ) ;\end{matrix}$$
wherein e′ᵢ stands for a respective node in the second similarity map; h^{t2}(e′ᵢ) stands for a respective vectorized representation of the respective node e′ᵢ prior to a t2-th step reiteration; h^{t2+1}(e′ᵢ) stands for an updated respective vectorized representation of the respective node e′ᵢ subsequent to the t2-th step reiteration; σ stands for a leaky relu activation function; N(e′ᵢ) stands for a set of nodes neighboring the respective node e′ᵢ; <h^{t2}(eᵢ′), h^{t2}(eₖ′)> stands for an inner product of h^{t2}(eᵢ′) and h^{t2}(eₖ′); Wₚ^{t2+1} stands for a parameter of a graph neural network for generating the vectorized representation; and α_{i,k}^{t2+1} stands for attention weights representing a link strength between node eᵢ′ and node eₖ′.

20. The method of claim 11, wherein the positive sample set is represented by {(m1ᵢ, m2ᵢ), i=1, . . . , K}, wherein m1ᵢ stands for an i-th macromolecule of the first type and m2ᵢ stands for an i-th macromolecule of the second type;
wherein training the model comprises minimizing a loss function:
$$L = - {\sum }_{i = I}^{K} log p ( 1 \vert dm {1}_{i} , dm {2}_{i} ) ;$$
wherein dm1ᵢ stands for vectorized representations of nodes in the first similarity map; dm2ᵢ stands for vectorized representations of nodes in the second similarity map; p(1|dm1ᵢ, dm2ᵢ) stands for a probability of interaction between a first respective vectorized representation of a node in the first similarity map and a second respective vectorized representation of a node in the second similarity map.

21. (canceled)

22. A neural network model for predicting macromolecule-macromolecule interaction, trained by the method of claim 11.
